(19) United States
(12) Patent Application Publication
(10) Pub. No.: US 2011/0126451 A1
(43) Pub. Date: Jun. 2, 2011

(54) INTEGRATED PROCESS FOR CONVERTING NATURAL GAS FROM AN OFFSHORE FIELD SITE TO LIQUEFIED NATURAL GAS AND LIQUID FUEL

(75) Inventors: Justin I-Ching Pan, Houston, TX (US); Lixin You, Sugar Land, TX (US)

(73) Assignee: Chevron U.S.A., Inc.

(21) Appl. No.: 12/627,949

(22) Filed: Nov. 30, 2009

(51) Int. Cl. C10L 1/18; F25J 1/00; F25J 3/00; F17C 9/00; C07C 27/00

(52) U.S. Cl. 44/451; 62/612; 62/648; 62/50.1; 518/700

(57) ABSTRACT

An integrated process and an apparatus for converting natural gas from an offshore field site to liquefied natural gas and to liquid fuel at an onshore site are disclosed. The process includes liquefying the natural gas and producing natural gas liquids using heat exchange at the offshore site. The liquefied natural gas can be transported to a market distribution location, and the natural gas liquids can be transported to the onshore site for further processing to liquid fuels. An air separation unit at the onshore site provides both liquefied nitrogen for use as coolant in the offshore heat exchange process as well as oxygen for use in an autothermal reformer at the onshore site. The natural gas liquids produced offshore can be fed to the autothermal reformer to generate synthesis gas which can be converted to liquid fuels.

### FIELD

The present invention relates to a process for converting hydrocarbon gas to useful products including liquefied natural gas and liquid fuel, and to a process for transporting such products. The present invention is particularly useful for converting and transporting stranded natural gas.

### BACKGROUND

There are numerous offshore oilfields having small volumes of associated natural gas as well as stranded small volume natural gas fields. For such small volumes of gas located remotely, finding an economical and environmentally sound means of disposing of the gas has proven to be a challenge. Gas reinjection is costly or impractical due to geophysical obstacles. Transporting gas via pipeline and as compressed natural gas is often uneconomical at great distances. Floating gas-to-liquids (GTL) plants and floating liquefied natural gas (LNG) plants are complicated and expensive to build. Environmental concerns make flaring increasingly unacceptable as a means of disposing of the gas.

It would be desirable to have a process for converting offshore associated gas and small volumes from gas fields into useful fuel products in an economical process which avoids complicated, large and heavy equipment offshore.

### SUMMARY

According to one embodiment, the invention relates to a process for converting natural gas from an offshore field site to liquefied natural gas and liquid fuel, comprising:
a) treating the natural gas at the field site to remove carbon dioxide;
b) reducing the temperature of the natural gas to form natural gas liquids;
c) further reducing the temperature of a portion of the natural gas in a natural gas liquefaction process to form liquefied natural gas;
d) transporting the liquefied natural gas from the field site to a market distribution site;
e) transporting the natural gas liquids from the field site to an onshore site having an air separation unit;
f) operating the air separation unit to generate a stream of oxygen and a stream of liquid nitrogen;
g) transporting the stream of liquid nitrogen generated by the air separation unit to the field site; and
h) utilizing the liquid nitrogen at the field site as a coolant in the natural gas liquefaction process.

According to another embodiment, the invention relates to an apparatus for converting natural gas from an offshore field site to liquefied natural gas and liquid fuel, comprising:
a) an acid gas removal unit for removing carbon dioxide from the natural gas at the field site;
b) a first heat exchanger capable of utilizing liquid nitrogen as a coolant for reducing the temperature of the natural gas to form natural gas liquids at the field site;
c) a second heat exchanger capable of utilizing liquid nitrogen as a coolant for further reducing the temperature of a portion of the natural gas liquids in a natural gas liquefaction process to form liquefied natural gas at the field site; and
d) an air separation unit at an onshore location capable of generating a stream of oxygen and a stream of liquid nitrogen.

### BRIEF DESCRIPTION OF THE DRAWINGS

FIG. 1 is a process flow diagram of an offshore process for converting natural gas from an offshore field site to liquefied natural gas, natural gas liquids and liquefied carbon dioxide.

FIG. 2 is a process flow diagram of an onshore process including air separation for generating oxygen and liquid nitrogen, and optionally further generating synthesis gas and converting the synthesis gas to liquid fuel.

### DETAILED DESCRIPTION OF THE EMBODIMENTS

An offshore process can be integrated with an onshore process wherein natural gas from an offshore field site is converted to liquefied natural gas at an offshore process site and to liquid fuel at an onshore process site.

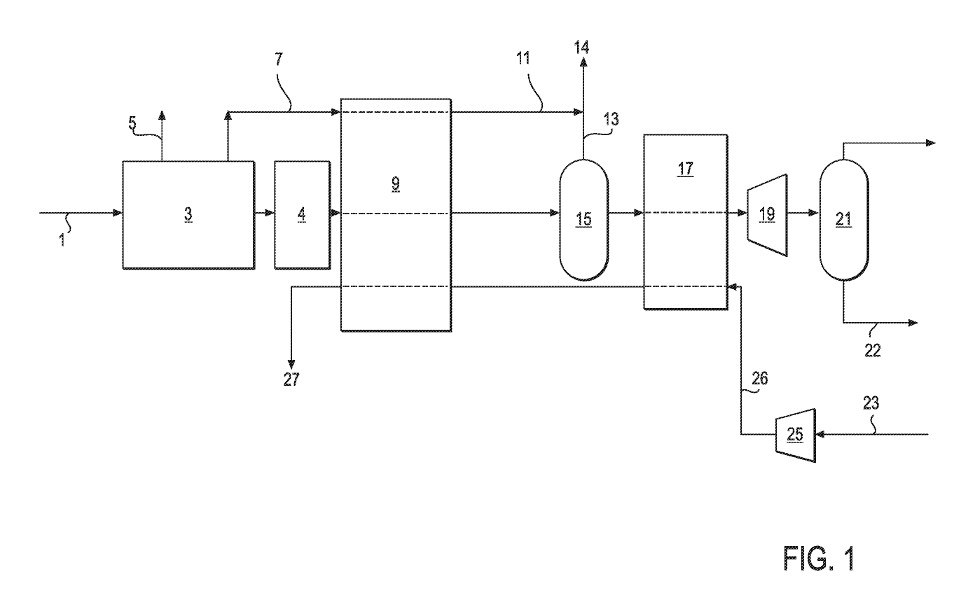

As shown in FIG. 1, natural gas 1 at the offshore field site is treated to remove acid components 5, also referred to as gas sweetening. Any known means 3 for removing acid components which is convenient for use at an offshore site is suitable. For instance, the natural gas can be contacted with an absorbent solution having an affinity for acid compounds such as carbon dioxide, hydrogen sulfide and mercaptans. Non-limiting examples of such solutions include amines, alkanolamines, polyamines, amino-acids, amino-acid alkaline salts, amides, ureas, alkali metal phosphates, carbonates and borates. One absorption process suitable for treating natural gas at an offshore site is disclosed in U.S. Patent Publication Number 2008/0210092 A1 hereby incorporated by reference.

The natural gas can alternatively be sweetened by contacting the gas with a combination of a gas permeable membrane followed by an absorbent solution as described above. The natural gas is brought into contact with one side of a permeable membrane and a sufficient positive pressure differential is maintained across the membrane such that the more permeable gaseous components of the mixture are driven from the feed side of the membrane to the permeate side. These more permeable components pass through the membrane at a higher rate than do other components of feed mixture which have lower permeabilities. Any membrane known for separating acid components from natural gas may be used, including for example cellulose ester and polyimide membranes. Suitable membranes and processes for using are disclosed in, for example, U.S. Pat. Nos. 4,130,403 and 4,589,896.

Optionally, the carbon dioxide 7 removed from the natural gas can subsequently be injected into an oil well for enhanced oil recovery (not shown), stored or sequestered in a geological formation (not shown), liquefied for transport to a market location as a liquefied carbon dioxide product (not shown), or liquefied in heat exchanger 9 to form liquefied carbon dioxide 11 and combined with natural gas liquids 13 to be transported as a combined NGL and liquefied carbon dioxide product 14 to the onshore site for further processing.

Natural gas from the acid gas removal unit 3 is subsequently treated such as by using molecular sieve dehydration process 4 in order to meet LNG specifications. For example, the molecular sieves may be crystalline metal alumina silicates having a three dimensional interconnecting network of silica and alumina tetrahedra. Those skilled in the art will appreciate that other types of molecular sieves which are capable of separating water from natural gas may also be used. Molecular sieves act as desiccants and are used as packing in two or more towers. In one such dehydration process, water is adsorbed from the gas by molecular sieves in one tower while the molecular sieves in another tower are offstream being regenerated. Hot gas is used to drive off the adsorbed water from the desiccant, after which the tower is cooled with an unheated gas stream. The onstream and offstream towers are switched before the onstream tower becomes water saturated. Mercury is also removed from the gas by known means.

The temperature of the sweetened and dehydrated natural gas is reduced at the field site using liquid nitrogen to separate natural gas liquids (NGL). The sweet dry gas is first cross exchanged with gaseous nitrogen in a heat exchanger 9, cooled to between about −60° C. and about −20° C., depending on feed composition then separately condensed into NGL 13 in a cold separator 15. The natural gas liquids include liquefied ethane, propane and other components including normal butane, isobutane, pentanes and higher hydrocarbons.

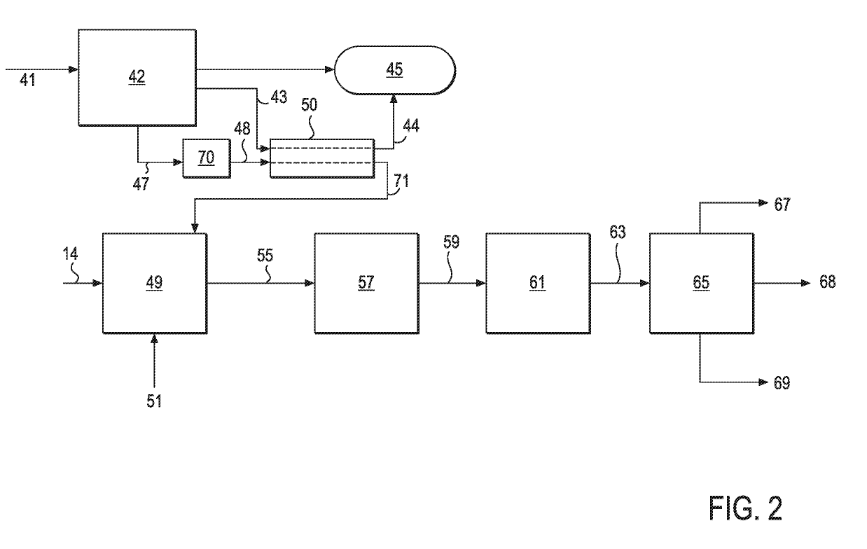

The temperature of a portion of the natural gas, the cold lean natural gas, containing predominately methane and ethane, is further reduced to between about −163° C. and about −161° C. to form liquefied natural gas (LNG) in a cryogenic heat exchanger 17 using liquid nitrogen (LIN) 26 as coolant. Liquid nitrogen is shipped from the onshore air separation unit (shown in FIG. 2) in an insulated LIN storage tank equipped with cryogenic LIN pumps 25 and delivered to the LNG heat exchanger 17. Alternatively, the LIN can be shipped to offshore storage and stored at atmospheric pressure prior to being pumped to a higher pressure for input into the LNG heat exchanger 17. The pressure is raised sufficiently to pass through both heat exchangers 17 and 9 sequentially. The warm nitrogen gas 27 from heat exchanger 9 is vented to the atmosphere.

The LNG 22 can then be transported from the field site to a market distribution site. Any marine vessel capable of storing and transporting LNG at cryogenic conditions is suitable. Nonlimiting examples of marine vessels suitable for storing and transporting LNG are disclosed in U.S. Pat. Nos. 3,680,323; 3,136,135; 2,933,902; 3,229,473; and 3,670,517.

The natural gas liquids 13 or combined NGL/liquefied carbon dioxide product 14 thus formed at the field site can be transported to an onshore site at ambient temperature as pressurized cargo on a marine vessel.

The onshore site, shown in FIG. 2, has an air separation unit 42, and further gas processing. According to one embodiment, the further gas processing includes a syngas generation unit 49 and a Fischer-Tropsch reactor 57 capable of converting syngas to liquid fuel.

The air separation unit (ASU) 42 generates a stream of oxygen 47 and a stream of nitrogen 43 using known technology. The air is first treated to remove any water and/or impurities that may be present then compressed using air compressors to ˜150 PSIG (not shown). The purified air then further undergoes compression to ˜300 PSIG and expansion and cooling prior to being fed to the ASU (not shown). The compressed air 41 is then sent to the ASU 42 for fractionation into nitrogen and oxygen. There are a variety of suitable processes by which air can be separated into oxygen and nitrogen. In a common type of air separation plant, air is partially or fully condensed within a bottom reboiler of a lower pressure column. The partially or fully condensed air is then rectified in the bottom of a higher pressure column. The rectification of the air produces a nitrogen rich tower overhead and oxygen rich column bottoms. Reflux for both the higher and lower pressure columns is produced by condensing a stream of the nitrogen rich tower overhead in an intermediate reboiler positioned within the lower pressure column. Examples of such processes may be found in U.S. Pat. Nos. 5,463,871 and 6,134,915.

Nitrogen from the ASU is in gas phase while the oxygen 47 from the ASU is in liquid phase. The liquid oxygen is pressurized to ˜400 PSIG as required by the gas to liquids (GTL) plant using a liquid oxygen (LOX) pumping system 70. High pressure LOX 48 is cross exchanged with gaseous nitrogen 43 in heat exchanger 50 to produce liquid nitrogen (LIN) 44 for shipment to the field site while vaporized oxygen 71 is supplied to the syngas generation unit 49 of the GTL plant. The LIN 44 can optionally be temporarily stored in onshore storage unit 45.

The stream of liquid nitrogen 44 is transported to the field site by cryogenic liquefied gas carriers (not shown) capable of maintaining nitrogen as a cryogenic liquid during transport.

NGL and liquid CO₂ from the field site can be disposed of by transporting offshore via a multigas carrier to the onshore GTL plant. NGL and CO₂ can be blended as a feed 14 to produce syngas. Alternatively, the NGL and liquid CO₂ can be sold. The NGL can alternatively be burned.

According to one embodiment, the stream of oxygen 71, steam 51, the combined NGL and carbon dioxide 14 can be fed to the onshore syngas generation unit 49 including autothermal reforming to generate syngas 55 (also referred to as synthesis gas) comprising a mixture of hydrogen and carbon monoxide. The syngas generation unit can have steam reforming, dry reforming and/or partial oxidation. Steam reforming, dry reforming, and partial oxidation proceed according to the following reactions:

Steam reforming: CₙHₘ+nH₂O+heat [symbol] nCO+(n+m/2)H₂ (1)

Dry Reforming: CₙHₘ+nCO₂+heat [symbol] 2nCO+m/2H₂ (2)

Partial Oxidation: CₙHₘ+n/2O₂ [symbol] nCO+m/2H₂+heat (3)

For a general discussion of steam reforming, dry reforming and partial oxidation, please refer to Harold G. Unardson, Industrial Gases in Petrochemical Processing 41-80 (1998), the contents of which are incorporated herein by reference. Prior to being fed to the syngas generation unit, the NGL can be treated for sulfur removal (not shown).

Syngas for use in the process can also be generated in a coal gasifier (not shown) or a biomass gasifier (not shown), which may be convenient if coal or biomass is available as a feedstock at the onshore site. Syngas can also be generated using boil-off gas from LNG storage tanks or other natural gas feeds. Alternatively, all or portion of LNG can be regasified and be used to generate syngas. High pressure steam generated by cooling of hot syngas can be used in a steam turbine to generate power or to drive air compressors directly in ASU unit 42.

According to one embodiment in which syngas is generated, the syngas is fed to the Fischer-Tropsch reactor 57 where it is converted to a hydrocarbon product including Fischer-Tropsch wax 59 by contact with a catalyst known for use in a Fischer-Tropsch (FT) process, such as cobalt, iron or ruthenium. A description of the FT process is found in Kirk-Othmer Encyclopedia of Chemical Technology, vol. 2, section 1.2 “Natural Gas Upgrading Via Fischer-Tropsch” in the chapter “Fuels, Synthetic, Liquid.” The product is upgraded through the use of a hydrocracking unit 61 which reduces the chain length of the wax component, thus producing a desired product, e.g., a middle distillate 63. The middle distillate is fed to a distillation column 65 for separation into desired end products, including, for example, naphtha 67, kerosene 68 and diesel 69.

Optionally, a hybrid catalyst may be used containing a FT catalyst component as well as an acid component in order to minimize wax production and thus minimize the need for hydrocracking after the FT synthesis reaction. An example of such a hybrid catalyst is given in U.S. patent application Ser. No. 12/343,534, the disclosure of which is hereby incorporated by reference.

Cooling of the Fischer-Tropsch reactor effluent is performed by any known means (not shown) including process heat exchange, boiler feedwater preheating, and/or using air and seawater cooling. Optionally, an expander (not shown) can be used to expand and cool the rich tail gas from the FT reactor to recover heavy liquids and/or produce electrical power. Any power generated can optionally be used in the air separation unit 42, e.g., to drive the compressor of the ASU. The lean tail gas can be routed to one of several areas depending on the plant configuration, including power generation, hydrogen generation or recycled to the FT reactor.

According to an alternate embodiment, rather than feeding the syngas 55 to an FT process, syngas is converted to methanol which is subsequently converted to gasoline in a methanol-to-gasoline (MTG) process (not shown). A description of the MTG process is found in Kirk-Othmer Encyclopedia of Chemical Technology, vol. 2, section 1.3 “Liquid Fuels via Methanol Synthesis and Conversion” in the chapter “Fuels, Synthetic, Liquid.”

According to yet another embodiment, syngas is not generated but rather the NGL produced offshore is delivered to a market distribution site. The oxygen produced by the onshore air separation unit 42 can be emitted to the atmosphere, or can be used as may be convenient at the onshore site. For instance, the oxygen can be fed to a coal gasification or biomass gasification process, or the oxygen can be fed to an oxyfuel process to produce concentrated CO₂ for sequestration (not shown).

The present process provides a simple offshore process in which the heat exchangers 9 and 17 necessary for gas liquefaction are the only major equipment needed. No complicated conventional refrigerant cycles for natural gas liquefaction such as cascade or mixed refrigerant are required. Natural gas can be separated into LNG, NGL components by virtue of having different boiling temperatures without the need for other gas separation processes. Both hydrocarbons and carbon dioxide will be recovered and converted, resulting in high thermal and carbon efficiency of the overall process. Both LNG and liquid fuels can be monetized from the same gas resource.

What is claimed is:

1. A process for converting natural gas from an offshore field site to liquefied natural gas and liquid fuel, comprising:
a) treating the natural gas at the field site to remove carbon dioxide;
b) reducing the temperature of the natural gas to form natural gas liquids;
c) further reducing the temperature of a portion of the natural gas to form liquefied natural gas;
d) transporting the liquefied natural gas from the field site to a market distribution site;
e) transporting the natural gas liquids from the field site to an onshore site having an air separation unit;
f) operating the air separation unit to generate a stream of oxygen and a stream of liquid nitrogen;
g) transporting the stream of liquid nitrogen generated by the air separation unit to the field site; and
h) utilizing the liquid nitrogen at the field site as a coolant in the natural gas liquefaction process.

2. The process of claim 1 wherein the natural gas at the field site is treated to remove carbon dioxide by means of an acid gas removal unit utilizing amine solvent having the capacity for amine regeneration.

3. The process of claim 1 wherein the natural gas at the field site is treated to remove carbon dioxide by means of a membrane.

4. The process of claim 1 wherein the carbon dioxide removed in step (a) is subsequently injected for enhanced oil recovery, stored in a geological formation, or liquefied for transport to a market location.

5. The process of claim 1 wherein the temperature is reduced in step (b) to between about −60° C. and about −20° C. to form the natural gas liquids.

6. The process of claim 1 wherein the temperature is reduced in step (c) to between about −163° C. and about −161° C. to form the liquefied natural gas.

7. The process of claim 1 wherein the natural gas liquids comprise ethane, propane and other components including normal butane, isobutane, pentanes and higher hydrocarbons.

8. The process of claim 1 wherein the onshore site further includes a syngas generation unit and a Fisher-Tropsch reactor capable of converting syngas to liquid fuel, and the process further comprises the steps of:
i) feeding the stream of oxygen and the natural gas liquids to the syngas generation unit to generate syngas comprising a mixture of hydrogen and carbon monoxide; and
j) converting the syngas in the Fisher-Tropsch reactor to liquid fuel.

9. The process of claim 8 wherein tail gas from the Fisher-Tropsch reactor is used to generate power.

10. The process of claim 8 wherein step (j) of converting occurs in the presence of a hybrid Fisher-Tropsch catalyst containing a FT catalyst component as well as an acid component.

11. The process of claim 1 wherein the process further comprises the steps of:
i) feeding the stream of oxygen and the natural gas liquids to a syngas generation unit to generate syngas comprising a mixture of hydrogen and carbon monoxide;
j) feeding the syngas to a methanol synthesis unit to produce methanol; and
k) converting the methanol to gasoline in a methanol to gasoline reactor capable of converting methanol to gasoline.

12. An apparatus for converting natural gas from an offshore field site to liquefied natural gas and liquid fuel, comprising:
a) an acid gas removal unit for removing carbon dioxide from the natural gas at the field site;
b) a first heat exchanger capable of utilizing liquid nitrogen as a coolant for reducing the temperature of the natural gas to between about −60° C. and about −20° C. to form natural gas liquids at the field site;
c) a second heat exchanger capable of utilizing liquid nitrogen as a coolant for further reducing the temperature of a portion of the natural gas in a natural gas liquefaction process to between about −163° C. and about −161° C. to form liquefied natural gas at the field site; and
d) an air separation unit at an onshore location capable of generating a stream of oxygen and a stream of liquid nitrogen.

13. The apparatus of claim 12 further comprising a syngas generation unit at the onshore location capable of generating a mixture of hydrogen and carbon monoxide.

14. The apparatus of claim 13 further comprising a Fisher-Tropsch reactor at the onshore location capable of converting syngas to liquid fuel.

15. The apparatus of claim 13 further comprising a methanol synthesis unit capable of converting syngas to methanol and a methanol to gasoline reactor capable of converting methanol to gasoline.

16. The apparatus of claim 12 further comprising a coal gasifier.

17. The apparatus of claim 12 further comprising a biomass gasifier.
